(19) United States
(12) Patent Application Publication
(10) Pub. No.: US 2021/0268057 A1
(43) Pub. Date: Sep. 2, 2021

(54) COMPOSITIONS AND METHODS FOR TREATING URINARY CONDITIONS

(71) Applicant: Innovus Pharmaceuticals, Inc., San Diego, CA (US)

(72) Inventor: Bassam Damaj, San Diego, CA (US)

(21) Appl. No.: 16/842,228

(22) Filed: Apr. 7, 2020

Related U.S. Application Data
(60) Provisional application No. 62/982,725, filed on Feb. 27, 2020.

(51) Int. Cl. A61K 36/54; A61K 47/36; A61K 47/02; A61K 36/11; A61K 36/185; A61K 9/48

(52) U.S. Cl. CPC A61K 36/54; A61K 47/36; A61K 9/4816; A61K 36/11; A61K 36/185; A61K 47/02

(57) ABSTRACT

Disclosed herein include methods, compositions, and kits suitable for use in treating or reducing a symptom of a urinary condition, such as, for example, overactive bladder and urinary incontinence. The method can comprise administration of a composition comprising herbal extract preparations.

### RELATED APPLICATIONS

This application claims the benefit under 35 U.S.C. § 119(e) of U.S. Provisional Patent Application Ser. No. 62/982,725, filed Feb. 27, 2020, the content of this related application is incorporated herein by reference in its entirety for all purposes.

### BACKGROUND

### Field

The present application relates to the fields of pharmaceutical chemistry, biochemistry and medicine. One aspect relates to the reduction and/or prevention of urinary incontinence or overactive bladder by administration of herbal compositions is provided herein.

### Description of the Related Art

The medical need is high for effective pharmacological treatments of urinary conditions (and symptoms thereof) such as overactive bladder (OAB), urgency, urge incontinence, (urinary) frequency, nocturia, stress incontinence and mixed urinary incontinence. This high medical need is a result of lack of efficacious pharmacological therapy coupled with high patient numbers.

Urinary incontinence (UI) with urinary urgency and/or frequency and Overactive Bladder (OAB) are common problems affecting one in five people in the United States. Bladder weakness affects 25% of reproductive age women, 50% of post-menopausal women, and 50%-75% of women in nursing homes. In men, 60% over the age of 60 experience benign prostate enlargement and associated OAB symptoms. Bladder problems remain under-diagnosed and under-reported.

Bladder control problems can occur for many reasons. Temporary bladder control problems may be caused by, for example, urinary tract infections, vaginal infections or irritation, constipation, and certain medicines. Longer lasting or chronic incontinence can be caused by, for example, both overactive and weak bladder muscles, obstruction from an enlarged prostate, damage to nerves that control the bladder from diseases such as multiple sclerosis or Parkinson's disease, or diseases such as arthritis that can make walking painful and slow. The basic types of bladder control problems include urinary urgency, urinary frequency, incontinence (bladder accidents with involuntary loss of urine) and nocturia (having to get out of bed at night for the toilet). Overactive bladder in some embodiments refers to both urinary frequency and urgency. There are multiple types of urinary incontinence, which include, for example, stress incontinence, urge incontinence, overflow incontinence, and functional incontinence. Stress incontinence occurs when urine leaks during exercise, coughing, sneezing, laughing, lifting heavy objects, or other body movements that put pressure on the bladder. This is the most common type of bladder control problem in younger and middle-age women. In some cases, it is related to the effects of childbirth. It may also begin around the time of menopause. In some embodiments, urge incontinence can happen when a person cannot hold his or her urine long enough to get to the toilet in time. Healthy people can have urge incontinence, but it is often found in people who have diabetes, stroke, Alzheimer's disease, Parkinson's disease, or multiple sclerosis. In some embodiments, overflow incontinence can happen when small amounts of urine leak from a bladder that is always full. A man can have trouble emptying his bladder if an enlarged prostate is blocking the urethra. Diabetes and spinal cord injury can also cause this type of incontinence. Functional incontinence can happen in many older people who have normal bladder control.

Medical treatments for bladder control problems, UI, and OAB can include physical and behavioral therapies, such as Kegel's pelvic floor exercises and bladder retraining, drug medications, devices such as catheters, and surgery. Current drug therapies include anticholinergics (with antispasmodic effects, e.g., oxybutinin), smooth muscle relaxants (antispasmodics), tricyclic antidepressants (e.g., imipramine), alpha-adrenergic antagonists, alpha-adrenergic agonists (e.g., phenylpropanolamine), prostaglandin synthesis inhibitors, calcium channel blockers and others. Unfortunately, most drug treatments are associated with unpleasant side effects, and these reduce patient compliance. Acetylcholine is the primary excitatory neurotransmitter involved in bladder emptying. Certain drugs commonly prescribed for urinary incontinence, such as oxybutynin hydrochloride, inhibit the muscarinic action of acetylcholine on smooth muscle, producing a direct antispasmodic action. These drugs relax the detrusor muscle. These medications also produce unwanted anticholinergic effects, such as dry mouth, blurred vision and constipation.

In some embodiments, overactive bladder is a condition that can be characterized by the sudden need to urinate. If that need results in the unintentional leakage of urine, the condition is called urge incontinence (“OAB wet”). Thus, urge incontinence falls within the general definition of OAB in some embodiments. In some embodiments, OAB can result from the sudden, involuntary contraction of the muscle in the wall of the urinary bladder. Approximately one-third of people with OAB also experience urge incontinence (“OAB wet”), while approximately two-thirds have OAB without urge incontinence (“OAB dry”). OAB, like urinary incontinence, is treated primarily with anticholinergic drugs (e.g., oxybutinin). These inhibit the neurotransmitter acetylcholine from attaching to the bladder muscle, and thereby reduce the frequency and intensity of contractions of the bladder. Unfortunately, adverse side effects of these drugs include dry mouth, dry eyes, constipation, and headache.

There are currently no medications that specifically target incontinence or OAB symptoms without having side effects elsewhere in the body. While some herbal approaches for bladder problems have been reported improve the tone and tissue strength of the bladder and surrounding area enough to be effective for bladder control problems, the timeframe for these herbal preparations to produce effective improvements in bladder control can be two to three months. In such instances, the length of time before effective results are experienced by a patient in need can result in distress and discomfort for the patient, as well as an expected reduction in patient compliance with the treatment. Herbal treatments that produce results within a shorter timeframe are warranted. Thus, a need exists for the identification of new herb-containing compositions that can provide faster and effective prevention or treatment of urinary conditions (and/or symptoms thereof), such as, for example, overactive bladder, nocturia, poor urinary stream and urinary incontinence. Further, there is a need for these new compositions without many of the unwanted side effects of currently available treatment options.

### SUMMARY

Disclosed herein include compositions. The composition can comprise: (i) a Lindera aggregate extract preparation of about 2 g to about 4 g dry weight equivalents; (ii) an Equisetum arvense extract preparation of about 2 g to about 4 g dry weight equivalents; (iii) a Crataeva magna extract preparation of about 4 g to about 8 g dry weight equivalents; (iv) about 100 mg to about 300 mg of a starch; and (v) about 5 mg to about 20 mg of a silicon compound. The composition can comprise: (i) a Lindera aggregate extract preparation of about 3 g dry weight equivalents; (ii) an Equisetum arvense extract preparation of about 3 g dry weight equivalents; (iii) a Crataeva magna extract preparation of about 6 g dry weight equivalents; (iv) about 185 mg of a starch; and (v) about 10 mg of a silicon compound.

In some embodiments, the composition is in a single unit dosage form. In some embodiments, the composition is in two or more unit dosage forms. In some embodiments, the composition is not formulated as an oral dosage unit. In some embodiments, the composition is formulated as an oral dosage unit whereby the oral dosage unit is in a form selected from a tablet, capsule or caplet. In some embodiments, the capsule comprises a vegetable capsule.

In some embodiments, the silicon compound comprises silica, silicon dioxide, talc, fumed silica, Zeolex, Neusilin SG2, Neusilin US2, precipitated silicate, sodium silicate, potassium silicate, magnesium silicate, calcium silicate, synthetic calcium silicate, zinc silicate, an aluminum silicate, sodium aluminosilicate, magnesium aluminum silicate, magnesium aluminum metasilicate, aluminum metasilicate, fumed silica, or any combination thereof. In some embodiments, the starch comprises wheat starch, corn starch, rice starch, oat starch, potato starch, maize starch, corn meal, buckwheat flour, millet flour, amaranth flour, quinoa flour, potato starch, sweet potato flour, tapioca starch, rice starch, rice flour, sorghum flour, bean flour, pea flour, pea starch, soy flour, chickpea flour, cowpea flour, lentil flour, bambara bean flour, lupin flour, peanut flour, chestnut flour, maize starch, corn meal, buckwheat flour, millet flour, amaranth flour, quinoa flour, potato starch, sweet potato flour, tapioca starch, rice starch, rice flour, sorghum flour, bean flour, pea flour, pea starch, soy flour, chickpea flour, cowpea flour, lentil flour, bambara bean flour, lupin flour, peanut flour, chestnut flour, or any combination thereof. In some embodiments, the starch comprises rice flour. In some embodiments, the silicon compound comprises silicon dioxide. In some embodiments, the composition does not comprise a component from Serenoa repens. In some embodiments, the composition does not comprise anhydrous colloidal silica.

In some embodiments, (i) the Lindera aggregate extract preparation is a root extract preparation; (ii) the Equisetum arvense extract preparation is a stem extract preparation; and/or (iii) the Crataeva magna extract preparation is a stem and bark extract preparation. In some embodiments, (i) the Lindera aggregate extract preparation is concentrated to an extract ratio of about 10:1; (ii) the Equisetum arvense extract preparation is concentrated to an extract ratio of about 10:1; and/or (iii) the Crataeva magna extract preparation is concentrated to an extract ratio of about 25:1.

In some embodiments, (i) the Lindera aggregate extract preparation is a non-standardized extract preparation; (ii) the Equisetum arvense extract preparation is a non-standardized extract preparation; and/or (iii) the Crataeva magna extract preparation is a non-standardized extract preparation. In some embodiments, (i) the Lindera aggregate extract preparation is a standardized extract preparation; (ii) the Equisetum arvense extract preparation is a standardized extract preparation; and/or (iii) the Crataeva magna extract preparation is a standardized extract preparation. In some embodiments, the Crataeva magna extract preparation is standardized to have lupeol content not less than about 1.5%. In some embodiments, the Crataeva magna extract preparation is standardized to have lupeol content of between about 0.50% and about 1.49%.

In some embodiments, (i) the total silicon concentration is less than about 32 mg dry weight equivalents per oral dosage unit; (ii) the phosphorus concentration is less than about 24 mg dry weight equivalents per oral dosage unit; (iii) the calcium concentration is less than about 16 mg dry weight equivalents per oral dosage unit; and/or (iv) the magnesium concentration is less than about 14 mg dry weight equivalents per oral dosage unit.

Disclosed herein include methods of treating or reducing a symptom of a urinary condition. The method can comprise administering an effective amount to a subject in need thereof any of the compositions disclosed herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include compositions. The composition can comprise: (i) a Lindera aggregate extract preparation of about 2 g to about 4 g dry weight equivalents; (ii) an Equisetum arvense extract preparation of about 2 g to about 4 g dry weight equivalents; (iii) a Crataeva magna extract preparation of about 4 g to about 8 g dry weight equivalents; (iv) about 100 mg to about 300 mg of a starch; and (v) about 5 mg to about 20 mg of a silicon compound. The composition can comprise: (i) a Lindera aggregate extract preparation of about 3 g dry weight equivalents; (ii) an Equisetum arvense extract preparation of about 3 g dry weight equivalents; (iii) a Crataeva magna extract preparation of about 6 g dry weight equivalents; (iv) about 185 mg of a starch; and (v) about 10 mg of a silicon compound.

Disclosed herein include methods of treating or reducing a symptom of a urinary condition. The method can comprise administering an effective amount to a subject in need thereof any of the compositions disclosed herein.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Spring Harbor Press (Cold Spring Harbor, N.Y. 1989). For purposes of the present disclosure, the following terms are defined below.

As used herein, a “subject” refers to an animal that is the object of treatment, observation or experiment. “Animals” include cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, a “patient” refers to a subject that has been treated by, or is being treated by, or will be treated by a medical professional, such as a Medical Doctor (i.e. Doctor of Allopathic medicine or Doctor of Osteopathic medicine) or a Doctor of Veterinary Medicine, to attempt to cure, or at least ameliorate the effects of, a particular disease or disorder or to prevent the disease or disorder from occurring in the first place.

As used herein, “administration” or “administering” refers to a method of giving a dosage of a composition disclosed herein to a vertebrate. There are provided herbal extract preparations comprising pharmaceutically active ingredients. In some embodiments, the pharmaceutically active ingredient is provided to a subject in the form of a herbal extract preparation.

As used herein, a “dosage” refers to the combined amount of the herbal extract preparations (e.g., the Lindera aggregate extract preparation, the Equisetum arvense extract preparation, and the Crataeva magna extract preparation).

As used herein, a “unit dosage” refers to an amount of therapeutic agent (e.g., the Lindera aggregate extract preparation, the Equisetum arvense extract preparation, and the Crataeva magna extract preparation) administered to a patient in a single dose.

As used herein, a “daily dosage” refers to the total amount of therapeutic agent (e.g., the Lindera aggregate extract preparation, the Equisetum arvense extract preparation, and the Crataeva magna extract preparation) administered to a patient in a day.

As used herein, “therapeutically effective amount” or “pharmaceutically effective amount” is meant an amount of therapeutic agent, which has a therapeutic effect. The dosages of a therapeutic agent which are useful in treatment are therapeutically effective amounts. Thus, as used herein, a therapeutically effective amount means those amounts of therapeutic agent which produce the desired therapeutic effect as judged by clinical trial results and/or model animal studies.

As used herein, a “therapeutic effect” relieves, to some extent, one or more of the symptoms of a disease or disorder. For example, a therapeutic effect may be observed by a reduction of the subjective discomfort that is communicated by a subject (e.g., reduced discomfort noted in self-administered patient questionnaire).

“Treat,” “treatment,” or “treating,” as used herein refers to administering a compound or a composition (e.g., a nutritional composition or a pharmaceutical composition) to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

The terms “prevent”, “preventing” and “prevention” as used herein refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

As used herein, a “synergistic” or “synergizing” effect can be such that the one or more effects of the combination compositions are greater than the one or more effects of each component alone, or they can be greater than the sum of the one or more effects of each component alone. The synergistic effect can be about, or greater than about 5, 10, 20, 30, 50, 75, 100, 110, 120, 150, 200, 250, 350, or 500% or even more than the effect on a subject with one of the components alone, or the additive effects of each of the components when administered individually. The effect can be any of the measurable effects described herein.

### Therapeutic Compositions

There are provided, in some embodiments, compositions. In some embodiments, the composition is or comprises a herb composition. In some embodiments, the composition is or comprises a herb extract preparation. In some embodiments, the composition is or comprises a standardized herb extract preparation. The form of the composition can vary. For example, the composition can be an oral composition, a foodstuff, a food supplement, a pharmaceutical composition, or any mixture thereof. In some embodiments, the composition is in a single unit dosage form. In some embodiments, the composition is in two or more unit dosage forms. In some embodiments, the composition is formulated for intravenous, intramuscular, rectal, or inhalation administration. In some embodiments, the composition comprises one or more pharmaceutically acceptable carriers, diluents or excipients. In some embodiments, the composition comprises a Lindera aggregate extract preparation, a Equisetum arvense extract preparation, and a Crataeva magna extract preparation. In some embodiments, the composition comprises one more additional components, such as, for example, a capsule (e.g. vegetable capsule), a starch (e.g., rice flour), and/or a silicon-containing substance (e.g., silicon dioxide). In several embodiments, the composition comprises a combination of various combination groups and individual ingredients. In some embodiments, the composition comprises, consists essentially of or consists of several or all of the following groups of ingredients:

(1) Lindera aggregate (e.g., Wu yao) extract preparation;

(2) Equisetum arvense (e.g., Horsetail) extract preparation;

(3) Crataeva magna (e.g., Three-leaf caper) extract preparation;

(4) a capsule (e.g. vegetable capsule);

(5) a starch (e.g., rice flour); and

(6) a silicon compound (e.g., silicon dioxide).

In some embodiments, only groups (1)-(3) above are provided. In some embodiments, groups (1)-(3) above are provided and the composition further comprises a selection of 1, 2, or 3 of groups (4)-(6). In some embodiments, the composition is in a single unit dosage form. In some embodiments, the composition is in two or more unit dosage forms. In some embodiments, the composition is formulated as single oral dosage unit. The composition is formulated as an oral dosage unit whereby the oral dosage unit is in a form selected from a tablet, capsule or caplet.

In some embodiments, one or more of groups (1)-(6) above are provided from about 1 mg to about 100,000 mg dry weight equivalents. In some embodiments, the amount of groups (1)-(6) above provided can be, or be about 0.1, 1, 10, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 10⁶, 10⁷, 10⁸, 10⁹, or a number or a range between any two of these values, mg dry weight equivalents. In some embodiments, the amount of groups (1)-(6) above provided can be at least, or be at most 0.1, 1, 1, 10, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 10⁶, 10⁷, 10⁸, or 10⁹, mg dry weight equivalents. In several embodiments, a therapeutic or effective amount of one or more of groups (1)-(6) above is included in the composition. A therapeutic or effective amount may be that which reduces the symptoms of overactive bladder and/or urinary incontinence.

In some embodiments, groups (1)-(3) above are provided as an extract preparation of one or more plant parts. As used herein, the term “plant part” refers to any part of a plant including, but not limited to, shoots, roots, stems, seeds, stipules, leaves, petals, flowers, eggs, bracts, branches, petioles, internodos, bark, pubescences, shoots, rhizomes, fronds, blades, pollen, stamens, fruits, or any combination thereof. In some embodiments, groups (1)-(3) above are provided as an extract preparation of 1, 2, 3, 4, 5, 6, 7, 8, 9, 10, 11, 12, 13, 14, 15, 16, or 17 different plant parts. In some embodiments, groups (1)-(3) above are provided as an extract preparation of all the parts of the plant that extend above-ground and/or below-ground. In some embodiments, group (1) is provided as a root extract preparation. In some embodiments, group (2) is provided as a stem extract preparation. In some embodiments, group (3) is provided as a stem extract preparation. In some embodiments, group (3) is provided as a bark extract preparation. In some embodiments, group (3) is provided as a stem and bark extract preparation.

In some embodiments, none of groups (1)-(3) above are provided as a standardized extract preparation. In some embodiments, one, two or all three of groups (1)-(3) above are provided as a standardized extract preparation. In some embodiments, one, two or all three of groups (1)-(3) above are provided as a standardized extract preparation standardized to contain not less than about, 0.000000001%, 0.00000001%, 0.0000001%, 0.000001%, 0.00001%, 0.0001%, 0.001%, 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, of one or more components based on the total weight (e.g., total dry weight) of the extract preparation or the composition as a whole. In some embodiments, one, two or all three of groups (1)-(3) above are provided as a standardized extract preparation standardized to contain not less than about, 0.000000001, 0.00000001, 0.0000001, 0.000001, 0.00001, 0.0001, 0.001, 0.01, 0.1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 10000, 100000, or a number or a range between any two of these values, mg dry weight equivalents of one or more components per extract preparation or per the composition as a whole. The standardization can be performed via any suitable compound(s), such as, for example, silicon, saponins, tannins, and/or lupeol.

In some embodiments, groups (1)-(3) above are provided as an extract preparation concentrated to an extract ratio of about, 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, 10000:1, or a number or a range between any two of the values. In some embodiments, groups (1)-(3) above are provided as an extract preparation with an extract ratio of least, or at most, 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, or 10000:1.

In some embodiments, groups (1)-(6) above can be provided in different amounts relative to each other. In some embodiments, two or more of groups (1)-(6) above are provided in equivalent ratios based on one or more of % m/m, % w/w, % m/v, % v/v, % m/w, % w/v, or dry weight equivalents. In some embodiments, two or more of groups (1)-(6) above are provided in non-equivalent ratios based on one or more of % m/m, % w/w, % m/v, % v/v, % m/w, % w/v, or dry weight equivalents. In some embodiments, the ratio of one or more of groups (1)-(6) above in the composition to one or more of groups (1)-(6) above in the composition can be, or be about, 1:1, 1:1.1, 1:1.2, 1:1.3, 1:1.4, 1:1.5, 1:1.6, 1:1.7, 1:1.8, 1:1.9, 1:2, 1:2.5, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100, 1:200, 1:300, 1:400, 1:500, 1:600, 1:700, 1:800, 1:900, 1:1000, 1:2000, 1:3000, 1:4000, 1:5000, 1:6000, 1:7000, 1:8000, 1:9000, 1:10000, or a number or a range between any two of the values, based on one or more of % m/m, % w/w, % m/v, % v/v, % m/w, % w/v, or dry weight equivalents. In some embodiments, groups (1)-(3) above are provided, based on dry weight equivalents, at a group (1):group (2):group (3) ratio of about 1:1:2.

As disclosed herein, compositions of particular ratios and/or amounts of ingredient groups (1)-(6) above can result in synergistic effects in reducing or preventing a urinary condition (or a symptom thereof), such as, for example, overactive bladder and/or urinary incontinence. These synergistic effects can be such that the one or more effects of the combination compositions are greater than the one or more effects of each ingredient alone at a comparable dosing level, or they can be greater than the predicted sum of the effects of all of the ingredients at a comparable dosing level, assuming that each ingredient acts independently. The synergistic effect can be about, or greater than about, 5, 10, 20, 30, 50, 75, 100, 110, 120, 150, 200, 250, 350, or 500% better than the effect of treating a subject with one of the ingredients alone, or the additive effects of each of the ingredients when administered individually. The effect can be any of the measurable effects described herein. The composition comprising a plurality of components can be such that the synergistic effect is an reduction in at least one of average daily frequency of urination, average nightly frequency of urination, total urinary incontinence episode, stress incontinence episodes, and urinary urgency episodes and that at least one of average daily frequency of urination, average nightly frequency of urination, total urinary incontinence episode, stress incontinence episodes, and urinary urgency episodes is reduced to a greater degree as compared to the sum of the effects of administering each component, determined as if each component exerted its effect independently, also referred to as the predicted additive effect herein. For example, if a composition comprising component (a) yields an effect of a 20% improvement in overactive bladder and/or urinary incontinence symptoms and a composition comprising component (b) yields an effect of 50% improvement in overactive bladder and/or urinary incontinence symptoms, then a composition comprising both component (a) and component (b) would have a synergistic effect if the combination composition's effect on overactive bladder and/or urinary incontinence symptoms was greater than 70%.

A synergistic combination composition can have an effect that is greater than the predicted additive effect of administering each ingredient of the combination composition alone as if each ingredient exerted its effect independently. For example, if the predicted additive effect is 70%, an actual effect of 140% is 70% greater than the predicted additive effect or is 1 fold greater than the predicted additive effect. The synergistic effect can be at least about 20, 50, 75, 90, 100, 150, 200 or 300% greater than the predicted additive effect. In some embodiments, the synergistic effect can be at least about 0.2, 0.5, 0.9, 1.1, 1.5, 1.7, 2, or 3 fold greater than the predicted additive effect.

In some embodiments, the synergistic effect of the combination compositions can also allow for reduced dosing amounts, leading to reduced side effects to the subject and reduced cost of treatment. Furthermore, the synergistic effect can allow for results that are not achievable through any other treatments. Therefore, proper identification, specification, and use of combination compositions can allow for significant improvements in the reduction and prevention of overactive bladder, urinary incontinence, nocturia, and/or poor urinary stream.

Disclosed herein include compositions comprising a silicon compound. The silicon compound can comprise silica, silicon dioxide, talc, fumed silica, Zeolex, Neusilin SG2, Neusilin US2, precipitated silicate, sodium silicate, potassium silicate, magnesium silicate, calcium silicate, synthetic calcium silicate, zinc silicate, an aluminum silicate, sodium aluminosilicate, magnesium aluminum silicate, magnesium aluminum metasilicate, aluminium metasilicate, fumed silica, or any combination thereof. For example, in some embodiments, the composition can contain silicon, such as in the form of silica, such as anhydrous silica. The additional silicon can assist with urogenital tissue support, strengthening and firmness. In some embodiments, the composition contains from about 10 mg dry weight equivalents to about 71 mg dry weight equivalents of total silicon per oral dosage unit. In some embodiments, the composition contains from about 15 mg dry weight equivalents to about 45 mg dry weight equivalents of total silicon per oral dosage unit. In some embodiments, the composition contains from about 28 mg dry weight equivalents to about 34 mg dry weight equivalents of total silicon per oral dosage unit. In some embodiments, the addition of a silicon compound (e.g., silicon dioxide) in the compositions disclosed herein results in unexpected synergistic effects in reducing or preventing overactive bladder and/or urinary incontinence. In some embodiments, the inclusion of the silicon compound unexpectedly yields one or more of the following additive or synergistic effects: improved shelf stability, increased efficacy, improved taste, improved bioavailability, more rapid disintegration after ingestion, quicker onset of action, and/or reduced side effects.

Disclosed herein include compositions comprising a starch. As used herein, the term “starch” shall be given its ordinary meaning and shall also refer to a carbohydrate consisting of a large number of glucose units joined by glycosidic bonds. In some embodiments, the starch does not contain gluten. The composition can comprise a starch selected from the group comprising maize starch, corn meal, buckwheat flour, millet flour, amaranth flour, quinoa flour, potato starch, sweet potato flour, tapioca starch, rice starch, rice flour, sorghum flour, bean flour, pea flour, pea starch, soy flour, chickpea flour, cowpea flour, lentil flour, bambara bean flour, lupin flour, peanut flour, chestnut flour, or any combination thereof. and combinations of these. For example, the starch can comprise rice flour. In some embodiments, the addition of a starch (e.g., rice flour) in the compositions disclosed herein results in unexpected synergistic effects in reducing or preventing overactive bladder and/or urinary incontinence. In some embodiments, the inclusion of the starch unexpectedly yields one or more of the following additive or synergistic effects: improved shelf stability, increased efficacy, improved taste, improved bioavailability, more rapid disintegration after ingestion, quicker onset of action, and/or reduced side effects.

In contrast to pre-existing herb-containing compositions, the compositions provided herein surprisingly can provide efficacy and efficiency much higher that the pre-existing compositions. For example, the compositions provided herein can result in an improvement of a urinary condition that is about at least two times, such as at least three times, four times, five times, or more, as fast as the pre-existing herb-containing compositions. For example, as compared to currently available compositions which may achieve improvement of a urinary condition in about three months, the compositions provided herein can achieve a comparable level of improvement in less than three months, such as less than two months, such as less than one month, such as less than two weeks. In some embodiments, the compositions disclosed herein can accomplish the improvement of a urinary condition between about two weeks and about two months, such as about two weeks, or such as about one month.

In some embodiments, the composition is an oral supplement included in a dry delivery system, e.g., tablet, dry powder, and dry meal replacement mixture. In some embodiments, the composition is an oral supplement included in a liquid delivery system, e.g., capsule, caplet, or beverage. In some embodiments, the composition is an oral supplement included in a controlled-release vehicle, e.g., tablet, caplet, and capsule. In some embodiments, it can be advantageous to formulate oral compositions in a dosage unit form for ease of administration and uniformity of dosage. Dosage unit forms described in some embodiments can refer to physically discrete units suited as unitary dosages for the subject to be treated; each unit containing a predetermined quantity of active composition calculated to produce the desired therapeutic effect in association with the suitable pharmaceutical carrier.

In some embodiments, the composition does not contain phosphorous. In some embodiments, the composition contains phosphorous. In some embodiments, the composition contains from about 5 mg dry weight equivalents of phosphorous to about 60 mg dry weight equivalents of phosphorous per oral dosage unit. In some embodiments, the composition contains from about 10 mg dry weight equivalents of phosphorous to about 50 mg dry weight equivalents of phosphorous per oral dosage unit. In some embodiments, the composition contains from about 20 mg dry weight equivalents of phosphorous to about 30 mg dry weight equivalents of phosphorous per oral dosage unit. In some embodiments, the phosphorous concentration is less than about 25 mg (e.g., 0, 0.000001, 0.00001, 0.0001, 0.001, 1, 2, 3, 4, 5, 6, 7, 8, 9, 10, 11, 12, 13, 14, 15, 16, 17, 18, 19, 20, 21, 22, 23, 24, 25 mg, or a number or a range between any two of these values) dry weight equivalents per oral dosage unit.

In some embodiments, the composition does not contain calcium. In some embodiments, the composition contains calcium. In some embodiments, the composition contains from about 1 mg dry weight equivalents of calcium to about 30 mg dry weight equivalents of calcium per oral dosage unit. In some embodiments, the composition contains from about 5 mg dry weight equivalents of calcium to about 25 mg dry weight equivalents of calcium per oral dosage unit. In some embodiments, the composition contains from about 10 mg dry weight equivalents of calcium to about 20 mg dry weight equivalents of calcium per oral dosage unit. In some embodiments, the calcium concentration is less than about 25 mg (e.g., 0, 0.000001, 0.00001, 0.0001, 0.001, 1, 2, 3, 4, 5, 6, 7, 8, 9, 10, 11, 12, 13, 14, 15, 16, 17, 18, 19, 20, 21, 22, 23, 24, 25 mg, or a number or a range between any two of these values) dry weight equivalents per oral dosage unit.

In some embodiments, the composition does not contain magnesium. In some embodiments, the composition contains magnesium. In some embodiments, the composition contains from about 1 mg dry weight equivalents of magnesium to about 30 mg dry weight equivalents of magnesium per oral dosage unit. In some embodiments, the composition contains from about 5 mg dry weight equivalents of magnesium to about 25 mg dry weight equivalents of magnesium per oral dosage unit. In some embodiments, the composition contains from about 10 mg dry weight equivalents of magnesium to about 20 mg dry weight equivalents of magnesium per oral dosage unit. In some embodiments, the magnesium concentration is less than about 25 mg (e.g., 0, 0.000001, 0.00001, 0.0001, 0.001, 1, 2, 3, 4, 5, 6, 7, 8, 9, 10, 11, 12, 13, 14, 15, 16, 17, 18, 19, 20, 21, 22, 23, 24, 25 mg, or a number or a range between any two of these values) dry weight equivalents per oral dosage unit.

### Herbal Extract Preparations

There are provided, in some embodiments, compositions comprising an extract preparation of Crateva magna (or “C. magna”) The bark of the tree has been reported to be used as a demulcent, antipyretic, sedative, alternative and tonic.

There are provided, in some embodiments, compositions comprising an extract preparation of Equisetum arvense (or “E. arvense”) (botanical synonyms and common names include, for example, Horsetail; Shave-grass; Bottle-brush; Paddock-pipes; Dutch Rushes; Pewterwort; Shavegrass; Pewterwort; Bottlebrush; Horsetail rush; Paddock-pipes; Dutch rushes; Mare's tail). Compared to the other herbs in the plant kingdom, horsetail can be very rich in silicon. The sterile stems can be harvested in summer and dried. The barren stems are useful as medicine, appearing after the fruiting stems have died down, and are used in their entirety, cut off just above the root. The herb can used either fresh or dried, but can be most efficacious when fresh in one embodiment. A fluid extract can prepared from it. The ashes of the plant can also be employed.

There are provided, in some embodiments, compositions comprising an extract preparation of Lindera aggregata (or “L. aggregata”) (botanical synonyms and common names include Lindera strychnifolia, Japanese evergreen spicebush, Chinese allspice, Evergreen Lindera, Kosterm, Uyaku (Japanese), Oyak (Korean)).

Herbs are useful in various forms, for example, as a homogenized mixture obtained by grinding or chopping an herb. The herbs are optionally subjected to processing such as extractions, for example by obtaining a filtrate by filtering or a supernatant by centrifugation. Known methods are readily used to extract a plant or any one or more portions of a plant (e.g., a leaf, root, seed, stem, and bark) as appropriate. In some embodiments, extracts that contain purified active ingredients are prepared. An isolated active ingredient can be an ingredient purified from C. magna, E. arvense, or L. aggregata that has activity to control (e.g., reduce) the symptoms of a urinary condition in a subject. Administration or use of an isolated active ingredient of another herb of the compositions herein, can be considered to be a use or administration of the herb itself. In some embodiments, any of the herbal preparations, including the C. magna, E. arvense, and L. aggregata, extract preparations, can be extracted using alcohol (e.g., 45-95% ethanol). Extraction can be from a solid matrix but can also be from liquids. Carbon dioxide (CO₂) can be commonly used as the super-critical fluid, sometimes modified with co-solvents such as ethanol or methanol. Extraction conditions for super-critical CO₂ may be above an advantageous temperature of 31° C. and an advantageous pressure of 74 bar.

In some embodiments, the composition contains from about 1,000 mg to about 6,000 mg dry weight equivalents C. magna stem/bark extract per oral dosage unit. In some embodiments, the composition contains from about 1,000 mg to about 4,000 mg dry weight equivalents C. magna stem/bark extract per oral dosage unit. In some embodiments, the composition contains from about 2,500 mg to about 3,500 mg dry weight equivalents C. magna stem/bark extract per oral dosage unit. In some embodiments, the composition contains about 3,000 mg dry weight equivalents C. magna stem/bark extract per oral dosage unit. A C. magna stem/bark extract is an extract prepared using both the stem parts and bark of the C. magna herb.

In some embodiments, the composition contains from about 1,000 mg to about 6,000 mg dry weight equivalents C. magna stem extract per oral dosage unit. In some embodiments, the composition contains from about 1,000 mg to about 4,000 mg dry weight equivalents C. magna stem extract per oral dosage unit. In some embodiments, the composition contains from about 2,500 mg to about 3,500 mg dry weight equivalents C. magna stem extract per oral dosage unit. In some embodiments, the composition contains about 3,000 mg dry weight equivalents C. magna stem extract per oral dosage unit.

In some embodiments, the composition contains from about 1,000 mg to about 6,000 mg dry weight equivalents C. magna bark extract per oral dosage unit. In some embodiments, the composition contains from about 1,000 mg to about 4,000 mg dry weight equivalents C. magna bark extract per oral dosage unit. In some embodiments, the composition contains from about 2,500 mg to about 3,500 mg dry weight equivalents C. magna bark extract per oral dosage unit. In some embodiments, the composition contains about 3,000 mg dry weight equivalents C. magna bark extract per oral dosage unit.

In some embodiments, to prepare the composition, the bark and/or stems of C. magna are isolated from the rest the C. magna plant and dried. The dried bark and stems of C. magna can be extracted using 70% ethanol/water. The liquid extract can be then concentrated to a ratio of 10:1. Maltodextrin can be used as an excipient. The final product, e.g., C. magna stem/bark extract, can be used in the composition as a brown to dark brown powder. In alternative embodiments, the liquid extract is then concentrated to a ratio of between about 25 and 35. Maltodextrin can be used as an excipient.

In some embodiments, the E. arvense extract preparation component of the composition is derived from the leaf of the E. arvense herb. In some embodiments, the E. arvense extract preparation component of the composition is derived from the stem of the E. arvense herb. In some embodiments, the E. arvense extract preparation component of the composition is derived from a mixture of plant parts of the E. arvense herb. In some embodiments, the E. arvense extract preparation component of the composition is derived from all the parts of the plant that extend above-ground. In some embodiments, the composition contains from about 1 mg to about 3,000 mg dry weight equivalents E. arvense extract preparation per oral dosage unit. In some embodiments, the composition contains from about 500 mg to about 2,500 mg dry weight equivalents E. arvense extract preparation per oral dosage unit. In some embodiments, the composition contains from about 1,000 mg to about 2,000 mg dry weight equivalents E. arvense extract preparation per oral dosage unit. In some embodiments, the composition contains from about 1,300 mg to about 1,600 mg dry weight equivalents E. arvense extract preparation per oral dosage unit. In some embodiments, the composition contains about 1,500 mg dry weight equivalents E. arvense stem extract per oral dosage unit.

In some embodiments, the L. aggregata extract preparation component of the composition is derived from the roots of the L. aggregata herb. In some embodiments, the L. aggregata extract preparation component of the composition is derived from the leaf and/or stem of the L. aggregata herb. In some embodiments, the L. aggregata extract preparation component of the composition is derived from a mixture of plant parts of the L. aggregata herb. In some embodiments, the L. aggregata extract preparation component of the composition is derived from all the parts of the plant that extend above-ground and/or below-ground. In some embodiments, the composition contains from about 1 mg to about 3,000 mg dry weight equivalents L. aggregata extract preparation per oral dosage unit. In some embodiments, the composition contains from about 500 mg to about 2,500 mg dry weight equivalents L. aggregata extract preparation per oral dosage unit. In some embodiments, the composition contains from about 1,000 mg to about 2,000 mg dry weight equivalents L. aggregata extract preparation per oral dosage unit. In some embodiments, the composition contains from about 1,300 mg to about 1,600 mg dry weight equivalents L. aggregata extract preparation per oral dosage unit. In some embodiments, the L. aggregata extract preparation can be present at a comparable, such as the same, concentration as the E. arvense preparation. In some embodiments, the composition contains about 1,500 mg dry weight equivalents L. aggregata root extract per oral dosage unit.

In some embodiments, the C. magna extract preparation can be extracted from the stem and/or bark of the plant, and the preparation is present at a concentration at least about 3,000 mg dry weight equivalents per oral dosage unit. That is, the starting material can be 3,000 mg of C. magna dry stem/bark. This starting material can be eventually concentrated during the manufacturing process to a ratio (“extract ratio”) of at least about 10 (i.e., 10:1), such as at least about 20, such as at least about 25, such as at least about 30, such as at least about 35, such as at least about 40. In some embodiments, the ratio is between about 25 and about 35. As an illustrative example, a ratio of 10 would be equivalent to 300 mg of C. magna preparation. Accordingly, 300 mg of C. magna stem/bark preparation (which is concentrated) is equivalent to 3,000 mg dry weight of C. magna stem/bark or 3,000 mg of C. magna dry stem/bark starting material. In some embodiments, the C. magna extract preparation is derived from the stem and/or bark parts of the C. magna herb, i.e., a C. magna stem/bark extract preparation.

The E. arvense extract preparation can be extracted from the stem of the plant, and the preparation is present at a concentration of at least about 1,500 mg dry weight equivalents per oral dosage unit. That is, the starting material is 1,500 mg of E. arvense herb. This starting material can be eventually concentrated during the manufacturing process to an extract ratio of at least about 5, such as at least about 8, such as at least about 10, such as at least about 15. As an illustrative example, a ratio of 4 or 5 would be equivalent to 375 mg or 300 mg, respectively, of E. arvense extract preparation. Thus, in the case of a concentration ratio of 5, for example, 300 mg of E. arvense extract preparation (which is concentrated) is equivalent to 1,500 mg dry weight of E. arvense herb or 1,500 mg of E. arvense dry herb starting material. In some embodiments, the E. arvense extract preparation is derived from the stem parts of the E. arvense herb, i.e., a E. arvense stem extract preparation.

The L. aggregata extract preparation can be extracted from the stem of the plant, and the preparation is present at a concentration of at least about 1,500 mg dry weight equivalents per oral dosage unit. That is, the starting material is 1,500 mg of L. aggregata herb. This starting material can be eventually concentrated during the manufacturing process to an extract ratio of at least about 5, such as at least about 8, such as at least about 10, such as at least about 15. As an illustrative example, a ratio of 4 or 5 would be equivalent to 375 mg or 300 mg, respectively, of L. aggregata extract preparation. Thus, in the case of a concentration ratio of 5, for example, 300 mg of L. aggregata extract preparation (which is concentrated) is equivalent to 1,500 mg dry weight of L. aggregata herb or 1,500 mg of L. aggregata dry herb starting material. In some embodiments, the L. aggregata extract preparation is derived from the root parts of the L. aggregata herb, i.e., a L. aggregata root extract preparation.

In some embodiments, one, two or all three of the herbal extract preparations are provided as a standardized extract preparation. In some embodiments, batch variation in the silicon content and/or flavonoid content expressed as isoquercetrin of E. arvense extract preparations can have negative effects on the biological activity of the composition described herein. This problem has been resolved in some embodiments by providing E. arvense extract preparations with optimized, standardized silicon content and/or flavonoid content expressed as isoquercetrin. Accordingly, there are provided, in some embodiments, compositions comprising a C. magna preparation, a L. aggregata preparation, and a standardized E. arvense extract preparation with a silicon content from about 3% to about 13% silicon based on total dry weight of the E. arvense preparation, wherein the composition is formulated as an oral dosage unit. Accordingly, for 1,500 mg dry weight of E. arvense herb or 1,500 mg of E. arvense dry herb starting material, which produces 300 mg of E. arvense extract preparation (which is concentrated), a silicon content from about 3% to about 13% would represent approximately 9 to 39 mg silicon.

In some instances, silicon is identified as a contributor to the biological activity of E. arvense herb. Non-standardized preparations of E. arvense herb can contain silicon from about 1.2% to about 6.9% silicon based on total dry weight of preparation. In some embodiments, batch variation in the silicon content of E. arvense extract preparations can have negative effects on the biological activity of the composition described herein. This problem can be resolved in one embodiment by providing an E. arvense extract preparation with optimized, standardized silicon content. Accordingly, in some embodiments, the silicon content of the E. arvense extract preparation in the preparation can be standardized. The use of a standardized preparation E. arvense herb can be advantageous because the inter-batch variation of silicon can be reduced, thus the composition described herein can yield more consistent preventative or therapeutic effect. In some embodiments, the E. arvense extract preparation is standardized to contain from about 3% silicon to about 13% silicon based on the total dry weight of the E. arvense extract preparation. In some embodiments, the E. arvense extract preparation is standardized to contain from about 5% silicon to about 10% silicon based on the total dry weight of the E. arvense extract preparation. In some embodiments, the E. arvense extract preparation is standardized to contain at least about 6% silicon based on the total dry weight of the E. arvense extract preparation.

In addition to silicon, E. arvense contains about 5 percent of a saponin, designated equisetonin, and several flavone glycosides (i.e., flavonoids) including isoquercetrin (i.e., isoquercitrin; Quercetin 3-O-β-D-glucopyrano side; 4H-1-B enzopyran-4-one, 2-(3,4-dihydroxy-phenyl)-3-((3-D-glucofuranosyloxy)-5,7-dihydroxy-), galuteolin, and equisetrin. Flavonoids, e.g., isoquercetrin, may have important pharmacological properties. Many flavonoids are diuretic, some are antispasmodic, anti-inflammatory, antiseptic and even antitumor. However, the predominant action of the flavonoids as a group can be on the vascular system. Without being bound by any particular theory, in some embodiments the flavone glycosides and the saponin combine to account for the diuretic action of E. arvense.

There are provided, in some embodiments, compositions comprising: a C. magna stem/bark preparation present at a concentration at least about 3,000 mg dry weight equivalents per oral dosage unit; an E. arvense stem extract preparation at a concentration of at least about 1,500 mg dry weight equivalents per oral dosage unit; and a L. aggregata root extract preparation at a concentration of at least about 1,500 mg dry weight equivalents per oral dosage unit.

There are provided, in some embodiments, compositions comprising a C. magna stem/bark preparation, an E. arvense stem extract preparation with a total flavonoid content from about 0.01% to about 3% total flavonoids based on the total dry weight of the E. arvense preparation, a L. aggregata root extract preparation; wherein the total flavonoid content is expressed as isoquercetrin and wherein the composition is formulated as an oral dosage unit.

In some embodiments, the E. arvense extract preparation can be a standardized E. arvense stem extract preparation. In some embodiments, the standardized E. arvense extract preparation further comprises a total flavonoid content from about 0.1% to about 2.5% total flavonoids based on the total dry weight of the E. arvense preparation and expressed as isoquercetrin. In some embodiments, the standardized E. arvense extract preparation comprises a total flavonoid content from about 0.5% to about 1.5% total flavonoids based on the total dry weight of the E. arvense preparation, wherein the total flavonoid content is expressed as isoquercetrin. In some embodiments, the standardized E. arvense extract preparation comprises a total flavonoid content from at least about 0.8% total flavonoids based on the total dry weight of the E. arvense preparation, wherein the total flavonoid content is expressed as isoquercetrin.

In some embodiments, the standardized E. arvense extract preparation further comprises a total flavonoid content from about 0.01% to about 3% total flavonoids based on the total dry weight of the E. arvense preparation, wherein the total flavonoid content is expressed as isoquercetrin. In some embodiments, the standardized E. arvense extract preparation further comprises a total flavonoid content from about 0.1% to about 2.5% total flavonoids based on the total dry weight of the E. arvense preparation and expressed as isoquercetrin. In some embodiments, the standardized E. arvense extract preparation further comprises a total flavonoid content from about 0.5% to about 1.5% total flavonoids based on the total dry weight of the E. arvense preparation, wherein the total flavonoid content is expressed as isoquercetrin. In some embodiments, the standardized E. arvense extract preparation further comprises a total flavonoid content from at least about 0.8% total flavonoids based on the total dry weight of the E. arvense preparation, wherein the total flavonoid content is expressed as isoquercetrin.

In some embodiments, batch variation in the total flavonoid content (expressed as isoquercetrin content) of E. arvense extract preparations can have negative effects on the biological activity of the composition described herein. This problem can been resolved in some embodiments by providing an E. arvense extract preparation with optimized, standardized total flavonoid content (expressed as isoquercetrin content). Accordingly, in some embodiments, the total flavonoid content (expressed as isoquercetrin content) of the E. arvense extract preparation in the preparation is standardized. The use of a standardized preparation E. arvense herb can be advantageous because the inter-batch variation of total flavonoid content (expressed as isoquercetrin content) is reduced, thus the composition provided herein can yield more consistent preventative or therapeutic effect. In some embodiments, the E. arvense extract preparation is standardized to contain from about 0.01% flavonoids to about 3% flavonoids based on the total dry weight of the E. arvense extract preparation, wherein the total flavonoids are expressed as isoquercetrin equivalents. In some embodiments, the E. arvense extract preparation is standardized to contain from about 0.1% flavonoids to about 2.5% flavonoids based on the total dry weight of the E. arvense extract preparation, wherein the total flavonoids are expressed as isoquercetrin equivalents. In some embodiments, the E. arvense extract preparation is standardized to contain from about 0.5% flavonoids to about 1.5% flavonoids based on the total dry weight of the E. arvense extract preparation, wherein the total flavonoids are expressed as isoquercetrin equivalents. In some embodiments, the E. arvense extract preparation is standardized to contain at least about 0.8% flavonoids based on the total dry weight of the E. arvense extract preparation, wherein the total flavonoids are expressed as isoquercetrin equivalents.

In some embodiments, the E. arvense extract preparation is standardized to organic silicon content by a solvent extraction process using raw material with a silicon content that met a minimum silicon content, e.g., 3% silicon. In some embodiments, the E. arvense extract preparation of the composition is derived from the stems of the E. arvense herb and standardized for silica content (i.e., E. arvense stem extract preparation). Briefly, stem parts of the E. arvense herb are removed from the plant and dried. They are then measured for a minimum of 2.5% silicon content via HPLC analysis before being accepted for the extraction process. An extract was obtained using 65% (v/v) ethanol/water extraction solvent. The extract was concentrated to a ratio of approximately 4:1. The extract is then tested again for minimum 3% silicon content via HPLC. The final extract dry concentrate appeared as a fine brown powder with a characteristic odor and taste.

In some embodiments, the E. arvense extract preparation is standardized to organic silicon by a solvent extraction process. Briefly, stem parts of the E. arvense herb are removed from the plant and dried. Morphological examination of the starting biomass (this includes both microscopic and macroscopic characteristics) can help facilitate using the correct species (e.g., an authenticated voucher specimen is stored on file for species identification). For example, an extract can be obtained using hot water (between about 50° C. and about 100° C.) as a solvent. The extract is concentrated to a ratio of approximately 5:1. The extract is then dried. The extract is tested for a minimum of approximately 3% silicon content via UV-Vis Spectrophotometry (silicon dioxide is used as a reference substance). In some embodiments, if the extract falls outside the desired standards above, it is titrated with a dried extract that had undergone the same process as above. The final extract dry concentrate can appear as a yellow-brown colored powder.

In some embodiments, the E. arvense extract preparation of the composition is derived from the stems of the E. arvense herb and standardized for total flavonoid content, i.e., E. arvense stem extract preparation.

In some embodiments, the E. arvense extract preparation is standardized to flavonoid (expressed as isoquercetrin) content by a solvent extraction process. Briefly, stem parts of the E. arvense herb can be removed from the plant and dried. They can be then identified by TLC. (e.g., using isoquercetrin as reference substance). Morphological examination of the starting biomass (e.g., microscopic and macroscopic characteristics) can help facilitate using the correct species (e.g., an authenticated voucher specimen was stored on file for species identification). An extract can be obtained using hot water (between about 50° C. and about 100° C.) as a solvent. The extract can be concentrated to a ratio of approximately 5:1. The extract can then be dried. The extract can be tested for a minimum of approximately 0.01% isoquercetrin via UV-Vis Spectrophotometry (using isoquercetrin as reference substance). If the extract falls outside the desired standards above, it can be titrated with a dried extract that had undergone the same process as above. The final extract dry concentrate can appear as a yellow-brown colored powder.

In some embodiments, the E. arvense extract preparation is standardized to organic silicon content and flavonoid content (expressed as isoquercetrin) using the methods described above.

In some embodiments, the Crateva magna extract preparation can be standardized, alone, or in addition to the other herbal preparations also being standardized, as described herein. For example, the Crateva magna extract preparation can be standardized to have at least one of the following based on total weight of the Crateva magna root extract preparation: (i) saponins not less than 25%; (ii) tannins not less than 2%; and/or (iii) lupeol not less than 1.5%.

In some embodiments, the C. magna extract preparation is standardized to contain not less than about 5% saponins based on the total dry weight of the C. magna extract preparation. In some embodiments, the C. magna extract preparation is standardized to contain not less than about 15% saponins based on the total dry weight of the C. magna extract preparation. In some embodiments, the C. magna extract preparation is standardized to contain not less than about 25% saponins based on the total dry weight of the C. magna extract preparation. In some embodiments, the C. magna extract preparation is standardized to contain not less than about 15% saponins based on the total dry weight of the C. magna extract preparation. In some embodiments, the C. magna extract preparation is standardized to contain not less than about 30% saponins based on the total dry weight of the C. magna extract preparation.

In some embodiments, the C. magna extract preparation is standardized to contain not less than about 0.5% tannins based on the total dry weight of the C. magna extract preparation. In some embodiments, the C. magna extract preparation is standardized to contain not less than about 1% tannins based on the total dry weight of the C. magna extract preparation. In some embodiments, the C. magna extract preparation is standardized to contain not less than about 2% tannins based on the total dry weight of the C. magna extract preparation. In some embodiments, the C. magna extract preparation is standardized to contain not less than about 4% tannins based on the total dry weight of the C. magna extract preparation. In some embodiments, the C. magna extract preparation is standardized to contain not less than about 5% tannins based on the total dry weight of the C. magna extract preparation.

In some embodiments, the C. magna extract preparation is standardized to contain not less than about 0.5% lupeol based on the total dry weight of the C. magna extract preparation. In some embodiments, the C. magna extract preparation is standardized to contain not less than about 1.5% lupeol based on the total dry weight of the C. magna extract preparation. In some embodiments, the C. magna extract preparation is standardized to contain not less than about 3% lupeol based on the total dry weight of the C. magna extract preparation. In some embodiments, the C. magna extract preparation is standardized to contain not less than about 5% lupeol based on the total dry weight of the C. magna extract preparation.

In some embodiments, all of the extract preparations are standardized. In some other embodiments, none of the extract preparations are standardized. The composition can comprise standardized C. magna extract preparation and not standardized extract preparations of E. arvense and L. aggregata. Alternatively, the composition can comprise standardized E. arvense extract preparation and not standardized extract preparations of C. magna and L. aggregata. Alternatively, the composition can comprise standardized L. aggregata extract preparation and not standardized extract preparations of C. magna and E. arvense. In some embodiments, the composition can comprise standardized extract preparations of C. magna and E. arvense and not standardized extract preparation of L. aggregata. Alternatively, the composition can comprise standardized extract preparations of C. magna and L. aggregata, and not standardized extract preparation of E. arvense. Alternatively, the composition can comprise standardized extract preparations of E. arvense and L. aggregata, and not standardized extract preparation of C. magna. The standardization can be accomplished via any suitable compound, such as, for example, silicon, saponins, tannins, lupeol, or any combination thereof. For example, in some embodiments, the Crateva magna extract preparation can be standardized to have at least one of the following based on total weight of the Crateva magna root extract preparation: (i) saponins not less than 25%; (ii) tannins not less than 2%; and/or (iii) lupeol not less than 1.5%.

### Administration of Therapeutic Compositions

Disclosed herein include herbal compositions for use in the treatment of urinary conditions (and/or symptoms thereof) such as overactive bladder, urgency, urge incontinence, frequency, nocturia, stress incontinence and mixed urinary incontinence, urgency, urge incontinence, frequency, nocturia, and/or mixed urinary incontinence. Disclosed herein include uses of a composition provided herein for the manufacture of a medicament for the treatment of urinary conditions (and/or symptoms thereof) such as overactive bladder, urgency, urge incontinence, frequency, nocturia, stress incontinence and mixed urinary incontinence, urgency, urge incontinence, frequency, nocturia, and/or mixed urinary incontinence. Disclosed herein include methods of treatment of urinary conditions (and/or symptoms thereof) such as overactive bladder, urgency, urge incontinence, frequency, nocturia, stress incontinence and mixed urinary incontinence, with a composition provided herein. There are provided, in some embodiments, methods of treating urinary conditions (and/or symptoms thereof) such as overactive bladder, urgency, urge incontinence, frequency, nocturia, stress incontinence and mixed urinary incontinence, urgency, urge incontinence, frequency, nocturia, and/or mixed urinary incontinence, comprising the administration to a patient in need of such treatment of a therapeutically effective amount of a composition provided herein. The urinary conditions (and/or symptoms thereof) that can be treated with the compositions provided herein include overactive bladder, urgency, urge urinary incontinence, urinary frequency, nocturia, mixed urinary incontinence, stress urinary incontinence, and genuine stress urinary incontinence. The terms “treating” or “treatment”, include the palliative, curative and prophylactic treatment of the above conditions, as well as complications arising from the above conditions and other associated conditions, including lower urinary tract symptoms associated with benign prostatic hypertrophy. The term “overactive bladder” refers to detrusor overactivity, i.e. overactivity of the bladder muscle. It includes the condition where the bladder contracts while it is filling. The term “urgency” is the complaint of a sudden compelling desire to pass urine, which is difficult to defer. The term “incontinence” or “urinary incontinence” refers to any involuntary leakage of urine. The term “urge incontinence” or “urge urinary incontinence” is the complaint of involuntary leakage accompanied by or immediately preceded by urgency. The term “frequency” is the complaint by the patient who considers that he/she voids too often.

The term “nocturia” is the complaint that the individual has to wake at night one or more times to void. The term “stress incontinence” or “stress urinary incontinence” (SUI) is the complaint of involuntary leakage upon anything that increases intraabdominal pressure, such as effort or exertion, sneezing or coughing. The term “mixed incontinence” or “mixed urinary incontinence” refers to the case when a patient suffers from more than one form of urinary incontinence, e.g. stress incontinence and urge incontinence. Overactive bladder can include symptoms of urgency, urge incontinence, frequency, nocturia, and/or mixed incontinence. There are provided, in some embodiments, compositions for the treatment of urinary conditions (and/or symptoms thereof) such as overactive bladder, urgency, urge incontinence, frequency, nocturia, stress incontinence and mixed urinary incontinence, urgency, urge incontinence, frequency, nocturia and/or mixed urinary incontinence. A subject in need of the presently described composition (and the administration thereof) can be one suffering any of the urogenital system disorders, including at least one of (i) urinary incontinence and (ii) overactive bladder symptoms. For example, the urogenital system disorder can include urinary incontinence, enuresis, benign prostatic hyperplasia, urinary calculi, cystitis, OAB, a urinary tract infection, and the like. Some embodiments herein provides compositions useful in a method of prophylaxis or treatment of disorders of BPH—e.g., urinary incontinence, enuresis (e.g., bed-wetting), benign prostatic hyperplasia, urinary calculi, cystitis, and urinary tract infection (or “UTI”).

Some embodiments disclosed herein include methods of treating or preventing a urinary condition comprising administering a composition disclosed herein to a subject in need thereof. In some embodiments, administering the compositions disclosed herein to a subject in need thereof treats or prevents a symptom of a urinary condition. Some such embodiments include therapeutic treatment, and some embodiments include prophylactic treatment.

The subject in need thereof can be a patient who is suffering from a urinary condition a subject that is suspected of or at the risk of developing a urinary condition. The subject may have, or may not have, symptoms of a urinary condition. In some embodiments, the subject does not have a urinary condition. In some embodiments, the subject has a urinary condition. The age for the subject in need thereof can vary. For example, the subject can be an adult, for example a middle-aged adult, or an elderly adult. In some embodiments, the subject is of the age of 40, 45, 50, 55, 60, 65, 70, 75, or more. For example, the subject can be a person at least 40 years old, or the subject can be a person at least 60 years old. The gender of the subject in need thereof can vary. In some embodiments, the subject is a female. In some embodiments, the subject is a male.

Some embodiments disclosed herein provide methods of treating or preventing a urinary condition (and/or symptoms thereof) by co-administering a Lindera aggregate extract preparation, an Equisetum arvense extract preparation, and/or a Crataeva magna extract preparation to a subject in need thereof. Some embodiments can include identifying a subject as having or at risk for developing a urinary condition (e.g., overactive bladder, urgency, urge incontinence, frequency, nocturia, stress incontinence, mixed urinary incontinence) prior to co-administering a Lindera aggregate extract preparation, an Equisetum arvense extract preparation, and/or a Crataeva magna extract preparation.

The co-administration of a Lindera aggregate extract preparation, an Equisetum arvense extract preparation, and/or a Crataeva magna extract preparation as described herein, can, in some embodiments, allow for a subject to experience a number of beneficial effects. These beneficial effects include the reduction of average daily frequency of urination, average nightly frequency of urination, total urinary incontinence episodes, stress incontinence episodes, and/or urinary urgency episodes. Compared to a baseline prior to treatment, these effects can result in an improvement of about or greater than about 5, 10, 15, 20, 30, 40, 50, 75, 100, 125, 150, 200, 250, 300, 400, or 500%. These amounts and/or levels can be maintained within 0, 1, 5, or 10% of the amounts and/or levels at the initiation of administration.

In some embodiments, the uses, methods and composition disclosed herein can reduce or prevent one or more symptoms of overactive bladder and/or urinary incontinence. For example, in some embodiments, the average daily frequency of urination is reduced in a patient receiving or received treatment by at least, or at least about, 5%, 10%, 20%, 30%, 40%, 50%, 60%, 70%, 80%, or 90% as compared to the patients received no treatment. In some embodiments, the total urinary incontinence episodes is reduced in a patient receiving or received treatment by at least, or at least about, 5%, 10%, 20%, 30%, 40%, 50%, 60%, 70%, 80%, or 90% as compared to the patients received no treatment. In some embodiments, the average nightly frequency of urination is reduced in a patient receiving or received treatment by at least, or at least about, 5%, 10%, 20%, 30%, 40%, 50%, 60%, 70%, 80%, or 90% as compared to the patients received no treatment. In some embodiments, the number of stress incontinence episodes is reduced in a patient receiving or received treatment by at least, or at least about, 5%, 10%, 20%, 30%, 40%, 50%, 60%, 70%, 80%, or 90% as compared to the patients received no treatment. In some embodiments, the number of urinary urgency episodes is reduced in a patient receiving or received treatment by at least, or at least about, 5%, 10%, 20%, 30%, 40%, 50%, 60%, 70%, 80%, or 90% as compared to the patients received no treatment. The compositions described herein can provide both short-term and long-term benefits according to several embodiments. Beneficial effects from use of the formulations described herein occur upon use, within hours of use, within 1-2 days, 3-4 days, about 7 days, about 14 days, or within about 3 weeks. In several embodiments, improvements in one or more symptoms of a urinary condition occur after administration of the composition and benefits continue over about 2-6 weeks, about 6-12 weeks, about 12-24 weeks, or about 24-52 weeks of use. In several embodiments, long lasting effects on the one or more symptoms of a urinary condition are achieved in less than 3 months.

### Methods of Administration of Therapeutic Compositions

Methods of treating urinary conditions by administering one or more compositions are disclosed herein. The compositions include the combination compositions described herein, such as combination compositions comprising a Lindera aggregate extract preparation, an Equisetum arvense extract preparation, and/or a Crataeva magna extract preparation. In general, an amount of a Lindera aggregate extract preparation, an Equisetum arvense extract preparation, and/or a Crataeva magna extract preparation in the levels sufficient to improve a urinary condition is administered for an effective period of time. The dosing of the combination compositions can be about, less than about, or more than about 1, 2, 3, 4, 5, 6, 7, 8, 9, 10 or more times daily. A subject can receive dosing for a period of about, less than about, or greater than about 1, 2, 3, 4, 5, 6, 7, 8, 9, 10, 11, 12, 13, 14, or more days, weeks or months. A unit dose can be chosen such that the subject is administered about 2 g to about 4 g dry weight equivalents of Lindera aggregate (e.g., Wu yao) extract preparation (e.g. about 2000 mg, 2100 mg, 2200 mg, 2350 mg, 2460 mg, 2500 mg, 2600 mg, 2750 mg, 2860 mg, 3000 mg, 3100 mg, 3390 mg, 3400 mg, 3520 mg, 3650 mg, 3700 mg, 3800 mg, 3900 mg, 4000 mg, or a number or a range between any two of these values), 2 g to about 4 g dry weight equivalents of Equisetum arvense (e.g., Horsetail) extract preparation (e.g. about 2000 mg, 2100 mg, 2200 mg, 2350 mg, 2460 mg, 2500 mg, 2600 mg, 2750 mg, 2860 mg, 3000 mg, 3100 mg, 3390 mg, 3400 mg, 3520 mg, 3650 mg, 3700 mg, 3800 mg, 3900 mg, 4000 mg, or a number or a range between any two of these values), and 4 g to about 8 g dry weight equivalents of Crataeva magna (e.g., Three-leaf caper) extract preparation (e.g. about 4000 mg, 4100 mg, 4200 mg, 4250 mg, 4260 mg, 4300 mg, 4390 mg, 4400 mg, 4520 mg, 4750 mg, 4900 mg, 5000 mg, 5100 mg, 5300 mg, 5530 mg, 5750 mg, 5900 mg, 6000 mg, 6100 mg, 6200 mg, 6250 mg, 6260 mg, 6300 mg, 6390 mg, 6400 mg, 6520 mg, 6750 mg, 6900 mg, 7000 mg, 7100 mg, 7300 mg, 7530 mg, 7750 mg, 7900 mg, 8000 mg, or a number or a range between any two of these values), about 100 mg to about 300 mg of a starch (e.g. about 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, or a number or a range between any two of these values), and about 5 mg to about 20 mg of a silicon compound (e.g. about 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, or a number or a range between any two of these values) daily. A unit dose can be a fraction of the daily dose, such as the daily dose divided by the number of unit doses to be administered per day. A unit dose can be a fraction of the daily dose that is the daily dose divided by the number of unit doses to be administered per day and further divided by the number of unit doses (e.g. tablets) per administration. The number of unit doses per administration may be about, less than about, or more than about 1, 2, 3, 4, 5, 6, 7, 8, 9, 10, or more. The number of doses per day may be about, less than about, or more than about 1, 2, 3, 4, 5, 6, 7, 8, 9, 10, or more. The number of unit doses per day may be determined by dividing the daily dose by the unit dose, and may be about, less than about, or more than about 1, 2, 3, 4, 5, 6, 7, 8, 9, 10, 11, 12, 13, 14, 15, 6, 17, 18, 19, 20, or more unit doses per day. For example, a unit dose can be about ½, ⅓, ¼, ⅕, ⅙, 1/7, ⅛, 1/9, or 1/10. A unit dose can be about one-third of the daily amount and administered to the subject three times daily. A unit dose can be about one-half of the daily amount and administered to the subject twice daily. A unit dose can be about one-fourth of the daily amount with two unit doses administered to the subject twice daily. A unit dose can comprise a capsule, such as, for example, an about 92 mg vegetable capsule comprising HPMC and water. The unit dose can comprise a capsule comprising about 420 mg of one or more herbal extract preparations. The unit dose can comprise a capsule comprising about 150 mg of Wu yao (Lindera aggregate root) extract preparation at an extract ratio of 10:1, about 150 mg of Horsetail (Equisetum arvense stem) extract preparation at an extract ratio of 10:1, about 120 mg of Three-leaf caper (Crataeva magna stem bark) extract preparation at an extract ratio of 25:1. The unit dose can comprise about 92 mg of a starch (e.g., rice flour). The unit dose can comprise about 5 mg of a silicon compound (e.g., silicon dioxide).

The compositions can be administered periodically. For example, the compositions can be administered one, two, three, four times a day, or even more frequent. The subject can be administered every 1, 2, 3, 4, 5, 6, or 7 days. In some embodiments, the compositions are administered three times daily. The administration can be concurrent with meal time of a subject. The period of treatment or diet supplementation can be for about 1, 2, 3, 4, 5, 6, 7, 8, or 9 days, 2 weeks, 1-11 months, or 1 year, 2 years, 5 years, or even longer. In some embodiments disclosed herein, the dosages that are administered to a subject can change or remain constant over the period of treatment. For example, the daily dosing amounts can increase or decrease over the period of administration.

The length of the period of administration and/or the dosing amounts can be determined by a physician, a nutritionist, or any other type of clinician. The period of time can be one, two, three, four or more weeks. In some embodiments, the period of time can be one, two, three, four, five, six or more months.

In some embodiments, the dosing level can be adjusted based on the subject's characteristics, such as weight, height, ethnicity, genetics, or baseline energy metabolism level.

In some embodiments, the compositions administered to a subject can be optimized for a given subject. For example, the ratio of Lindera aggregate extract preparation, Equisetum arvense extract preparation, and/or Crataeva magna extract preparation or the particular components in a combination composition can be adjusted. The ratio and/or particular components can be selected after evaluation of the subject after being administered one or more compositions with varying ratios of Lindera aggregate extract preparation, Equisetum arvense extract preparation, and/or Crataeva magna extract preparation, or varying combination composition components.

As disclosed herein, the Lindera aggregate extract preparation, Equisetum arvense extract preparation, and/or Crataeva magna extract preparation do not have to be administered in the same composition to perform the claimed methods. For example, separate capsules, pills, mixtures, etc. of Lindera aggregate extract preparation, Equisetum arvense extract preparation, and/or Crataeva magna extract preparation may be administered to a subject to carry out the claimed methods. The administration of Lindera aggregate extract preparation, Equisetum arvense extract preparation, and/or Crataeva magna extract preparation may be at the same time or at different times provided that effective concentrations of components of the Lindera aggregate extract preparation, Equisetum arvense extract preparation, and/or Crataeva magna extract preparation are both found in the subject at the same time. In some embodiments, administration of the Lindera aggregate extract preparation, Equisetum arvense extract preparation, and/or Crataeva magna extract preparation is at the same time, preferably in a single composition, in order to facilitate the compliance of the subject to adhere to a schedule of administration.

In some embodiments, the dosing regimen of the compositions disclosed herein is administered for a period of time, which time period can be, for example, from at least about 1 week to at least about 4 weeks, from at least about 4 weeks to at least about 8 weeks, from at least about 4 weeks to at least about 12 weeks, from at least about 4 weeks to at least about 16 weeks, or longer. The dosing regimen of the compositions disclosed herein can be administered three times a day, twice a day, daily, every other day, three times a week, every other week, three times per month, once monthly, substantially continuously or continuously.

The formulation, route of administration and dosage for the compositions disclosed herein can be chosen by the individual physician in view of the patient's condition. Typically, the dose range of the composition administered to the patient can be from about 0.1 to about 4000 mg/kg of the patient's body weight. The dosage may be a single one or a series of two or more given in the course of one or more days, as is needed by the patient. In instances where human dosages for the compositions have been established for at least some condition, the present disclosure will use those same dosages, or dosages that are between about 0.1% and about 5000%, more preferably between about 25% and about 1000% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Although the exact dosage will be determined on a drug-by-drug basis, in most cases, some generalizations regarding the dosage can be made. In some embodiments, the composition is administered 1 to 4 times per day. As will be understood by those of skill in the art, in certain situations it may be necessary to administer the compositions disclosed herein in amounts that exceed, or even far exceed, the above-stated, preferred dosage range in order to effectively and aggressively treat particularly aggressive diseases or infections. In some embodiments, the compositions disclosed herein are administered for a period of continuous therapy, for example for a week or more, or for months or years.

### Compositions for Administration

The combination compositions can be formulated for oral administration in the form of a tablet, a capsule, or any other form described herein. The compositions can be administered to a subject orally or by any other methods. Methods of oral administration include, in some embodiments, administering the composition as a liquid, a solid, or a semisolid that can be taken in the form of a dietary supplement or a foodstuff.

In some embodiments, the administration of Lindera aggregate extract preparation, Equisetum arvense extract preparation, and/or Crataeva magna extract preparation is oral. The Lindera aggregate extract preparation, Equisetum arvense extract preparation, and/or Crataeva magna extract preparation may be formulated together with suitable carriers such as starch, sucrose or lactose in tablets, pills, dragees, capsules, solutions, liquids, slurries, suspensions and emulsions. The Lindera aggregate extract preparation, Equisetum arvense extract preparation, and/or Crataeva magna extract preparation may be administered separately or together, provided that the total amount of a Lindera aggregate extract preparation, a Equisetum arvense extract preparation, and/or a Crataeva magna extract preparation is an effective amount in combination per day to have a substantial impact on the symptoms of overactive bladder and/or urinary incontinence.

According to a further aspect, some embodiments relate to an oral composition comprising a Lindera aggregate extract preparation, a Equisetum arvense extract preparation, and/or a Crataeva magna extract preparation in a foodstuff, in a food supplement, or in a pharmaceutical preparation.

In some embodiments, a food composition for human consumption is supplemented by the above composition. For example, the food composition can be, or comprise, a nutritional complete formula, a dairy product, a chilled or shelf stable beverage, a powdered beverage, a mineral or purified water, a liquid drink, a soup, a dietary supplement, a meal replacement, a nutritional bar, a confectionery, a milk, a fermented milk product, a yoghurt, a milk based powder, an enteral nutrition product, an infant formula, an infant nutritional product, a cereal product or a fermented cereal-based product, an ice cream, a chocolate, coffee, a culinary product such as mayonnaise, tomato puree, salad dressings, a pet food, or any combination thereof.

For ingestion, many embodiments of oral compositions and in particular of food supplements are contemplated herein. They are formulated by means of the usual methods for producing sugar-coated tablets, pills, pastes, gums, gelatin capsules, gels, emulsions, tablets, capsules or drinkable solutions or emulsions, which can then be taken directly with water or by any other known means.

The nutritional supplement for oral administration may be in capsules, gelatin capsules, soft capsules, tablets, sugar-coated tablets, pills, pastes or pastilles, gums, or drinkable solutions or emulsions, syrups or gels, with a dose of about 0.001 to 100% of the primary composition, which can then be taken directly with water or by any other known means. This supplement may also include a sweetener, a stabilizer, an additive, a flavoring or a colorant. A supplement for cosmetic purpose can additionally comprises a compound active with respect to the skin. Methods for preparing them are common knowledge.

Also, the formulation as described above may be incorporated into any other forms of food supplements or of enriched foods, for example food bars, or compacted or non-compacted powders. Methods for preparing them are common knowledge.

The food composition or food supplement may also include a sweetener, a stabilizer, an antioxidant, an additive, a flavoring or a colorant. The composition may also contain synthetic or natural bioactive ingredients such as amino acids, fatty acids, vitamins, minerals, carotenoids, polyphenols, etc. that can be added either by dry or by wet mixing to said composition before pasteurization and/or drying. According to some embodiments, the composition disclosed herein can be used cosmetically. By “cosmetic use” is meant a non-therapeutic use which may improve the aesthetic aspect or comfort of the skin, coat and/or hair of humans or pets.

In some embodiments, a pharmaceutical composition can be administered for prophylactic and/or therapeutic treatments. In therapeutic applications, compositions are administered to a patient already suffering from a disease, as described herein under, in an amount sufficient to cure or at least partially arrest the symptoms of the disease and its complications. An amount adequate to accomplish this is defined as “a therapeutically effective dose”. Amounts effective for this will depend on the severity of the disease and the weight and general state of the patient. In prophylactic applications, compositions disclosed herein are administered to a patient susceptible to or otherwise at risk of a particular disease. Such an amount is defined to be “a prophylactic effective dose”. In this use, the precise amounts again depend on the patient's state of health and weight.

The compositions disclosed herein are, in some embodiments, administered with a pharmaceutically acceptable carrier, the nature of the carrier differing with the mode of administration, for example, enteral, oral and topical (including ophthalmic) routes. The desired formulation can be made using a variety of excipients including, for example, pharmaceutical grades of mannitol, lactose, starch, magnesium stearate, sodium saccharin, cellulose, magnesium carbonate. This composition may be a tablet, a capsule, a pill, a solution, a suspension, a syrup, a dried oral supplement, a wet oral supplement.

Furthermore, in some embodiments the composition can be intravenously administered in any suitable manner. For administration via intravenous infusion, the composition is preferably in a water-soluble non-toxic form. Intravenous administration is particularly suitable for hospitalized patients that are undergoing intravenous (IV) therapy. For example, the composition can be dissolved in an IV solution (e.g., a saline or glucose solution) being administered to the patient. Also, the composition can be added to nutritional IV solutions, which may include amino acids and/or lipids. The amounts of the composition to be administered intravenously can be similar to levels used in oral administration. Intravenous infusion may be more controlled and accurate than oral administration.

As disclosed herein, the combination of a Lindera aggregate extract preparation, a Equisetum arvense extract preparation, and/or a Crataeva magna extract preparation can be formulated for administration in a pharmaceutical composition comprising a physiologically acceptable surface active agents, carriers, diluents, excipients, smoothing agents, suspension agents, film forming substances, coating assistants, or a combination thereof. In some embodiments, the combination of a Lindera aggregate extract preparation, a Equisetum arvense extract preparation, and/or a Crataeva magna extract preparation are formulated for administration with a pharmaceutically acceptable carrier or diluent. The combination of a Lindera aggregate extract preparation, a Equisetum arvense extract preparation, and/or a Crataeva magna extract preparation can be formulated as a medicament with a standard pharmaceutically acceptable carrier(s) and/or excipient(s) as is routine in the pharmaceutical art. The exact nature of the formulation will depend upon several factors including the desired route of administration. Typically, combination of a Lindera aggregate extract preparation, a Equisetum arvense extract preparation, and/or a Crataeva magna extract preparation are formulated for oral, intravenous, intragastric, intravascular or intraperitoneal administration. Standard pharmaceutical formulation techniques may be used, such as those disclosed in Remington's The Science and Practice of Pharmacy, 21st Ed., Lippincott Williams & Wilkins (2005), incorporated herein by reference in its entirety.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman' s: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerin, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with a composition can be determined by the way the composition is to be administered.

The compositions described herein are preferably provided in unit dosage form. As used herein, a “unit dosage form” is a composition that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the composition. The amount of carrier employed in conjunction with the composition is sufficient to provide a practical quantity of material for administration per unit dose of the composition. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et ah, Pharmaceutical Dosage Forms: Tablets (1989); and Ansel, Introduction to Pharmaceutical Dosage Forms 8th Edition (2004).

Various oral dosage forms can be used, including such solid forms as tablets, capsules, and granules. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Other compositions useful for attaining systemic delivery can be in, for example, sublingual, buccal and/or nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

For topical use, creams, ointments, gels, solutions or suspensions, etc., containing the compositions disclosed herein are employed. Topical formulations may generally be comprised of a pharmaceutical carrier, co-solvent, emulsifier, penetration enhancer, preservative system, and emollient.

For intravenous administration, the compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HC1, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al, Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In some embodiments, the compositions are provided in solution ready to administer parenterally. In some embodiments, the compositions are provided in a solution that is further diluted prior to administration. In embodiments that include administering the compositions described herein and another agent(s) (for example, a silicon, a starch, or a combination thereof), the combination may be provided to caregivers as a mixture, or the caregivers may mix the two agents prior to administration, or the two agents may be administered separately.

In some embodiments, a combination composition comprises a Lindera aggregate extract preparation, a Equisetum arvense extract preparation, a Crataeva magna extract preparation, and one or more additional ingredients. An additional ingredient may serve one or more functions. In some embodiments, an additional ingredient accounts for about, less than about, or more than about 0.1%, 0.5%, 1%, 2%, 3%, 4%, 5%, 6%, 7%, 8%, 9%, 10%, 15%, 20%, 25%, 30%, 40%, 50%, 60%, 70%, 80%, 90%, 95%, or more of the mass or volume of the combination composition. Non-limiting examples of additional ingredients include sweeteners, bulking agents, stabilizers, acidulants, preservatives, binders, lubricants, disintegrants, fillers, solubilizers, coloring agents (such as fruit juice and vegetable juice), and other additives and excipients known in the art. In some embodiments, a combination composition comprises one or more (e.g. 1, 2, 3, 4, 5, 6, 7, 8, 9, 10, 15, 20, or more) sweeteners. Examples of sweeteners include, but are not limited to, sucrose, fructose, dextrose, maltose, lactose, high fructose corn syrup solids, invert sugar, sugar alcohols, sorbitol, saccharin, cyclamates, sweeteners derived from stevia, sweeteners derived from momordica grosvenorii, sweeteners derived from mogrosides, acesulfame K, L-aspartyl-L-phenylalanine lower alkyl ester sweeteners, L-aspartyl-D-alanine amide sweeteners, L-aspartyl-D-serine amide sweeteners, L-aspartyl-L-1-hydroxymethylalkaneamide sweeteners, L-aspartyl-1-hydroxy ethyalkaneamide sweeteners, L-aspartyl-D-phenylglycine ester and amide sweeteners, rebaudioside A, rebaudioside B, rebaudioside C, rebaudioside D, rebaudioside E, rebaudioside F, dulcoside A, dulcoside B, rubusoside, stevia, stevia extract, stevioside, mogroside IV, mogroside V, siamenoside, monatin and their salts (monatin SS, RR, RS, SR), curculin, glycyrrhizic acid and its salts, thaumatin, monellin, mabinlin, brazzein, hemandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, cyclocarioside I, sucralose, potassium acesulfame, aspartame, alitame, saccharin, neohesperidin dihydrochalcone, cyclamate, neotame, N—[N-[3-(3-hydroxy-4-methoxyphenyl)propyl]-L-a-aspartyl]L-phenylalanine-1-methyl ester, N—[N-[3-(3-hydroxy-4-methoxyphenyl)-3-methylbutyl]-L-a-aspartyl]-L-phenylalanine-1-methyl ester, N—[N-[3-(3-methoxy-4-hydroxyphenyl)propyl]-L-a-aspartyl]-L-phenylalanine-1-methyl ester, salts thereof, or combinations thereof. In some embodiments, the sweetener is a polyol additive, such as a sugar alcohol, erythritol, maltitol, mannitol, sorbitol, lactitol, xylitol, inositol, isomalt, propylene glycol, glycerol (glycerine), threitol, galactitol, palatinose, reduced isomalto-oligosaccharides, reduced xylo-oligosaccharides, reduced gentio-oligosaccharides, reduced maltose syrup, or reduced glucose syrup.

In some embodiments, a combination composition comprises one or more (e.g. 1, 2, 3, 4, 5, 6, 7, 8, 9, 10, 15, 20, or more) bulking agents. Non-limiting examples of bulking agents include guar gum, locust bean gum, cassia gum, pectin from botanical sources, high molecular weight carboxymethylcellulose, carrageenan, alginate, and xanthane. In some embodiments, one or more bulking agents may be added to enhance the viscosity of a liquid formulation.

In some embodiments, a combination composition comprises one or more (e.g. 1, 2, 3, 4, 5, 6, 7, 8, 9, 10, 15, 20, or more) stabilizers. Non-limiting examples of stabilizers include pectin, polysaccharide hydrolysates comprising dextrin, agar, can-ageenan, tamarind seed polysaccharides, angelica gum, karaya gum, xanthan gum, sodium alginate, tragacanth gum, guar gum, locust bean gum, pullulan, gellan gum, gum arabic, carboxymethylcellulose, and propylene glycol alginate ester. In some embodiments, one or more stabilizers are added to the combination composition to enhance the shelf-life of the combination composition. In general, shelf-life refers to the amount of time the container and composition therein can be held at ambient conditions (approximately room temperature, e.g. about 18-28° C.) or less, without degradation of the composition and/or container occurring to the extent that the composition cannot be used in the manner and for the purpose for which it was intended. In some embodiments, the combination composition has a shelf life of about, less than about, or more than about 1, 2, 3, 4, 5, 6, 7, 8, 9, 10, 11, 12, 14, 30, 60, 90, or more days; or about, less than about, or more than about 1, 2, 3, 4, 5, 6, 7, 8, 9, 10, 11, 12, or more months or years. In some embodiments, the combination composition remains non-perishable for a period of time after opening a container containing the composition. In general, perishability refers to degradation to an extent that the composition cannot be used in the manner and purpose for which it was designed. In some embodiments, the combination composition remains non-perishable for about, less than about, or more than about 1, 2, 3, 4, 5, 6, 7, 8, 9, 10, 11, 12, 18, 24, 30, 36, 48, 60, 72, 90, or more hours or days after opening; or about, less than about, or more than about 1, 2, 3, 4, 5, 6, 8, 10, 11, 12, or more months or years after opening. In some embodiments, the combination composition remains nonperishable for a period of time at room temperature (e.g. about 18-28° C.). In some embodiments, the combination composition remains non-perishable for a period of time upon refrigeration, such as storage below about 20° C., 15° C., 10° C., 5° C., 4° C., 3° C., 2° C., 1° C., 0° C., −1° C., −2° C., −3° C., −4° C., −5° C., −10° C., −20° C., or lower.

In some embodiments, a combination composition comprises one or more (e.g. 1, 2, 3, 4, 5, 6, 7, 8, 9, 10, 15, 20, or more) acidulants. Non-limiting examples of acidulants include C2-C30 carboxylic acids, substituted hydroxyl C1-C30 carboxylic acids, benzoic acid, substituted benzoic acids (e.g. 2,4-dihydroxybenzoic acid), substituted cinnamic acids, hydroxyacids, substituted hydroxybenzoic acids, substituted cyclohexyl carboxylic acids, tannic acid, lactic acid, tartaric acid, citric acid, gluconic acid, glucoheptonic acids, adipic acid, hydroxycitric acid, malic acid, fruitaric acid (a blend of malic, fumaric, and tartaric acids), fimaric acid, maleic acid, succinic acid, chlorogenic acid, salicylic acid, creatine, glucosamine hydrochloride, glucono delta lactone, caffeic acid, bile acids, acetic acid, ascorbic acid, alginic acid, erythorbic acid, polyglutamic acid, and their alkali or alkaline earth metal salt derivatives thereof.

In some embodiments, a combination composition comprises one or more (e.g. 1, 2, 3, 4, 5, 6, 7, 8, 9, 10, 15, 20, or more) preservatives. Non-limiting examples of preservatives include sorbic acid, benzoic acid, and salts thereof, including (but not limited to) calcium sorbate, sodium sorbate, potassium sorbate, calcium benzoate, sodium benzoate, potassium benzoate, and mixtures thereof.

In some embodiments, the compositions can be a food product, for example a snack bar, or a beverage, comprising a Lindera aggregate extract preparation, a Equisetum arvense extract preparation, and/or a Crataeva magna extract preparation. For example, the snack bar can be a chocolate bar, a granola bar, or a trail mix bar. In some embodiments, the present dietary supplement or food compositions are formulated to have suitable and desirable taste, texture, and viscosity for consumption. Any suitable food carrier can be used in the present food compositions. Food carriers of the compositions described herein include practically any food product. Examples of such food carriers include, but are not limited to food bars (granola bars, protein bars, candy bars, etc.), cereal products (oatmeal, breakfast cereals, granola, etc.), bakery products (bread, donuts, crackers, bagels, pastries, cakes, etc.), beverages (milk-based beverage, sports drinks, fruit juices, alcoholic beverages, bottled waters), pastas, grains (rice, corn, oats, rye, wheat, flour, etc.), egg products, snacks (candy, chips, gum, chocolate, etc.), meats, fruits, and vegetables. In some embodiments, food carriers employed herein can mask the undesirable taste (e.g., bitterness). Where desired, the food composition presented herein exhibit more desirable textures and aromas than that of any of the components described herein. For example, liquid food carriers can be used to obtain the present food compositions in the form of beverages, such as supplemented juices, coffees, teas, shakes (e.g., milk shakes), smoothies, and the like. In some embodiments, solid food carriers can be used to obtain the present food compositions in the form of meal replacements, such as supplemented snack bars, pasta, breads, and the like. In some embodiments, semi-solid food carriers can be used to obtain the present food compositions in the form of gums, chewy candies or snacks, and the like.

In some embodiments, the compositions provided herein comprise no microbes or pharmaceutically acceptable low numbers of microbes. In some embodiments, the disclosed compositions contain less than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 10000, 100000, or a number or a range between any two of these values, colony forming units per gram. For example, the disclosed compositions can contain less than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 10000, 100000, or a number or a range between any two of these values, colony forming units of yeast, mold, coliforms, E. coli, S. aureus, and/or Salmonella per gram.

In some embodiments, the compositions provided herein comprise no heavy metals or contaminants (e.g., pesticide residues) or pharmaceutically acceptable low numbers of heavy metals or contaminants. For example, in some embodiments, the levels of arsenic, cadmium, lead, and/or mercury in the disclosed compositions can be less than about, 0.000000001, 0.00000001, 0.0000001, 0.000001, 0.00001, 0.0001, 0.001, 0.01, 0.1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, or a number or a range between any two of these values, ug/oral dosage unit (e.g., ug/capsule).

The herb-containing compositions disclosed herein can be used alone or further formulated with pharmaceutically acceptable compositions, vehicles, or adjuvants with a favorable delivery profile (i.e., suitable for delivery to a subject, particularly one in need thereof). Such compositions can comprise the herb-containing composition and a pharmaceutically acceptable carrier. “Pharmaceutically acceptable carrier” in some embodiments is intended to include any and all solvents, dispersion media, coatings, antibacterial and antifungal compositions, isotonic and absorption delaying compositions, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, Ringer's solutions, dextrose solution, and 5% human serum albumin. The use of such media and compositions for pharmaceutically active substances is well known in the art. Except insofar as any conventional media or composition is incompatible with the active composition, use thereof in the compositions is contemplated. Supplementary active compositions can also be incorporated into the compositions.

Oral compositions disclosed herein can include an inert diluent or an edible carrier. Oral compositions disclosed herein can be enclosed in gelatin capsules, caplets or compressed into tablets. For the purpose of oral therapeutic administration, the herb-containing composition can be incorporated with excipients and used in the form of tablets, troches, or capsules. Oral compositions can also be prepared using a fluid carrier for use as a mouthwash, wherein the composition in the fluid carrier is applied orally and swished and expectorated or swallowed. Pharmaceutically compatible binding compositions, and/or adjuvant materials can be included as part of the composition. The tablets, pills, capsules, troches and the like can contain any of the following ingredients, or compositions of a similar nature: a binder such as microcrystalline cellulose, gum tragacanth or gelatin; an excipient such as starch or lactose, a disintegrating composition such as alginic acid, Primogel, or corn starch; a lubricant such as magnesium stearate or Sterotes; a glidant such as colloidal silicon dioxide; a sweetening composition such as sucrose or saccharin; or a flavoring composition such as peppermint, methyl salicylate, or orange flavoring. The herb-containing compositions provided herein can also be formulated as a topical cream for transdermal or transmucosal administration.

In some embodiments, the herb-containing compositions are prepared with carriers that will protect the composition against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

### Kits

Also provided herein are kits comprising one or more compositions described herein, in suitable packaging, and may further comprise written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. A kit may comprise one or more unit doses described herein. In some embodiments, a kit comprises about, less than about, or more than about 1, 2, 3, 4, 5, 6, 7, 8, 9, 10, 11, 12, 13, 14, 15, 16, 17, 18, 19, 20, 30, 31, 60, 90, 120, 150, 180, 210, or more unit doses. Instructions for use can comprise dosing instructions, such as instructions to take 1, 2, 3, 4, 5, 6, 7, 8, 9, 10, or more unit doses 1, 2, 3, 4, 5, 6, 7, 8, 9, 10, or more times per day. For example, a kit may comprise a unit dose supplied as a tablet, with each tablet package separately, multiples of tablets packaged separately according to the number of unit doses per administration (e.g. pairs of tablets), or all tablets packaged together (e.g. in a bottle). As a further example, a kit may comprise a unit dose supplied as a bottled drink, the kit comprising 1, 2, 3, 4, 5, 6, 7, 8, 9, 10, 11, 12, 13, 14, 24, 28, 36, 48, 72, or more bottles.

The kit can further contain another agent. In some embodiments, the Lindera aggregate extract preparation, the Equisetum arvense extract preparation, and the Crataeva magna extract preparation are provided as separate compositions in separate containers within the kit. In some embodiments, the Lindera aggregate extract preparation, the Equisetum arvense extract preparation, and the Crataeva magna extract preparation are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including but not limited to, physicians, nurses, pharmacists, formulary officials, and the like. Kits can also, in some embodiments, be marketed directly to the consumer.

In some embodiments, a kit can comprise a multi-day supply of unit dosages. The unit dosages can be any unit dosage described herein. The kit can comprise instructions directing the administration of the multi-day supply of unit dosages over a period of multiple days. The multi-day supply can be a one-month supply, a 30-day supply, or a multi-week supply. The multi-day supply can be a 90-day, 180-day, 3 -month or 6-month supply. The kit can include packaged daily unit dosages, such as packages of 1, 2, 3, 4, or 5 unit dosages. The kit can be packaged with, for example, other dietary supplements, vitamins, and meal replacement bars, mixes, and beverages.

### Terminology

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A composition, comprising:
(i) a Lindera aggregate extract preparation of about 2 g to about 4 g dry weight equivalents;
(ii) an Equisetum arvense extract preparation of about 2 g to about 4 g dry weight equivalents;
(iii) a Crataeva magna extract preparation of about 4 g to about 8 g dry weight equivalents;
(iv) about 100 mg to about 300 mg of a starch; and
(v) about 5 mg to about 20 mg of a silicon compound.

2. The composition of claim 1, comprising:
the Lindera aggregate extract preparation of about 3 g dry weight equivalents;
the Equisetum arvense extract preparation of about 3 g dry weight equivalents;
the Crataeva magna extract preparation of about 6 g dry weight equivalents;
(iv) about 185 mg of the starch; and
(v) about 10 mg of the silicon compound.

3. The composition of claim 1, wherein the composition is in a single unit dosage form.

4. The composition of claim 1, wherein the composition is in two or more unit dosage forms.

5. The composition of claim 1, wherein the composition is not formulated as an oral dosage unit.

6. The composition of claim 1, wherein the composition is formulated as an oral dosage unit whereby the oral dosage unit is in a form selected from a tablet, capsule or caplet.

7. The composition of claim 6, wherein the capsule comprises a vegetable capsule.

8. The composition of claim 1, wherein the silicon compound comprises silica, silicon dioxide, talc, fumed silica, Zeolex, Neusilin SG2, Neusilin US2, precipitated silicate, sodium silicate, potassium silicate, magnesium silicate, calcium silicate, synthetic calcium silicate, zinc silicate, an aluminum silicate, sodium aluminosilicate, magnesium aluminum silicate, magnesium aluminum metasilicate, aluminium metasilicate, fumed silica, or any combination thereof.

9. The composition of claim 1, wherein the starch comprises wheat starch, corn starch, rice starch, oat starch, potato starch, maize starch, corn meal, buckwheat flour, rice flour, pea flour, pea starch, soy flour, chickpea flour, cowpea flour, lentil flour, bambara bean flour, lupin flour, peanut flour, chestnut flour, millet flour, amaranth flour, quinoa flour, sweet potato flour, tapioca starch, sorghum flour, bean flour, lupin flour, peanut flour, chestnut flour, or any combination thereof.

10. The composition of claim 1, wherein the starch comprises rice flour.

11. The composition of claim 1, wherein the silicon compound comprises silicon dioxide.

12. The composition of claim 1, wherein the composition does not comprise a component from Serenoa repens.

13. The composition of claim 1, wherein the composition does not comprise anhydrous colloidal silica.

14. The composition of claim 1, wherein
(i) the Lindera aggregate extract preparation is a root extract preparation;
(ii) the Equisetum arvense extract preparation is a stem extract preparation; and/or
(iii) the Crataeva magna extract preparation is a stem and bark extract preparation.

15. The composition of claim 1, wherein
(i) the Lindera aggregate extract preparation is concentrated to an extract ratio of about 10:1;
(ii) the Equisetum arvense extract preparation is concentrated to an extract ratio of about 10:1; and/or
(iii) the Crataeva magna extract preparation is concentrated to an extract ratio of about 25:1.

16. The composition of claim 1, wherein
(i) the Lindera aggregate extract preparation is a non-standardized extract preparation;
(ii) the Equisetum arvense extract preparation is a non-standardized extract preparation; and/or
(iii) the Crataeva magna extract preparation is a non-standardized extract preparation.

17. The composition of claim 1, wherein
(i) the Lindera aggregate extract preparation is a standardized extract preparation;
(ii) the Equisetum arvense extract preparation is a standardized extract preparation; and/or
(iii) the Crataeva magna extract preparation is a standardized extract preparation.

18. The composition of claim 1, wherein the Crataeva magna extract preparation is standardized to have lupeol content not less than about 1.5%.

19. The composition of claim 1, wherein the Crataeva magna extract preparation is standardized to have lupeol content of between about 0.50% and about 1.49%.

20. The composition of claim 6, wherein
(i) the total silicon concentration is less than about 32 mg dry weight equivalents per oral dosage unit;
(ii) the phosphorus concentration is less than about 24 mg dry weight equivalents per oral dosage unit;
(iii) the calcium concentration is less than about 16 mg dry weight equivalents per oral dosage unit; and/or
(iv) the magnesium concentration is less than about 14 mg dry weight equivalents per oral dosage unit.

21. A method of treating or reducing a symptom of a urinary condition, the method comprising administering an effective amount to a subject in need thereof the composition of claim 1.
